(19) United States
(12) Patent Application Publication
(10) Pub. No.: US 2004/0126447 A1
(43) Pub. Date: Jul. 1, 2004

(54) Extract of Dioscorea sp. and the medical uses thereof

(76) Inventor: Rong-Tsun Wu, Taipei City (TW)

Correspondence Address: WHYTE HIRSCHBOECK DUDEK S C, 555 EAST WELLS STREET, SUITE 1900, MILWAUKEE, WI, 53202, US

(21) Appl. No.: 10/335,278

(22) Filed: Dec. 31, 2002

(51) Int. Cl. A61K 35/78

(52) U.S. Cl. 424/773

(57) ABSTRACT

The present invention provides extract fractions of Dioscorea sp. prepared by the method according to the present invention, which enhance the proliferation and differentiation of bone marrow cells, and provides a method for treatment of osteoporosis and a method for alleviation of side effects caused by chemotherapy.

### BACKGROUND OF THE INVENTION

1. Field of the Invention

The present invention relates to an oral composition comprising an active extract of Dioscorea sp., in particular to an oral composition capable of enhancing the proliferation and differentiation of cells, and of alleviating the side effects induced by chemotherapy. The present invention provides a method for treatment of osteoporosis and a method for alleviation of side effects caused by chemotherapy.

2. Description of the Related Art

Dioscorea, also known as “wild yam,” is a member of the monocotyledonous family Dioscoreaceae, which is distributed in the tropical and subtropical regions. There are about 650 species in the world, of which 93 species and 9 varieties are found in China, and 14 species and 5 varieties are found in Taiwan.

Dioscorea is one of the very important pharmaceutical plants used in traditional Chinese medicine, and the medicinal effects thereof have been studied for years. In 1936, Tsukamoto et al isolated diosgenin, a steroid saponin of Dioscorea, from Dioscoreacea family of plants and then used it as raw material for rapid synthesis of medicinal steroids. In a study by Aradhana, Rao Ar. Kale R K. in 1992, it was indicated that diosgenin promotes the growth of epithelial cells of rat mammary glands. In Biochemical & Biophysical Research Communications 207(1):398-404, February/1995, J. L. Beneytout et al reported that Diosgenin induces morphological and biochemical changes characteristic of megakaryocyte cells when diosgenin is added to human erythroleukemia (HEL) cell cultures and, thus, diosgenin can be used as a megakaryotic differentiation inducer of HEL cells. In Life Sciences 59(11):147-57, 1996, a steroid extract of Dioscorea was indicated to possess significant activities as an anti-oxidant to modify serum lipid levels.

Dehydroepiandrosterone (DHEA) has similar chemical structure to diosgenin, and is known to have anti-cancer, anti-oxidation, and anti-diabetic effects, as well as the effect of regulation of bone mass. The serum levels of DHEA gradually decrease as age increases, and are related to aging. It was speculated from various studies that the diosgenin extract of Dioscorea might be converted into DHEA in the human body and thus supplements the DHEA which decreases with aging. However, these studies are only conducted on old people taking diosgenin present in the Dioscorea to investigate if diosgenin could reduce the over-oxidation of serum lipids, lower the triglycerides in blood serum and increase HDL level while decreasing the over-oxidation damage of LDL.

Concerning the effect of DHEA on regulation of bone mass, in Life Sciences 62(1):59-68, 1998, Ben A. A. Scheven et al reported that DHEA and its sulfate derivative (DHEA-S) failed on their own to exert direct, independent significant effects on the growth and differentiation of human osteoblastic cells, but treating the cells in conjunction with a bone cell modulating agent, 1,25(OH)₂D₃, resulted in enhancement of specific alkaline phosphatase (ALP) activity, which is the specific marker of maturing osteoblastic cells. This study shows that the effects of DHEA/DHEA-S on osteoblastic cell growth and differentiation are likely to be mediated via an effect on 1,25(OH)₂D₃-induced change in bone cells.

In accordance with the present invention, it was found that the methanol extract of Dioscorea sp. and the further extracted fractions possess biological activity on cell regeneration. Specifically, it was found that the methanol extract of Dioscorea sp. and the further extracted fractions per se, without the presence of any bone cell modulating agent, stimulate the proliferation and differentiation of the osteoprogenitor cells so as to supplement the osteoprogenitor cells in the bone and promote the maturity of osteoblastic cells and mineralization of osteoblastic cells, thereby achieving bone repair, restoration and reproduction and in turn preventing and treating osteoporosis. Moreover, the extract of Dioscorea sp. not only stimulates the proliferation and differentiation of hematopoietic stem cells in bone marrow in the presence of GM-CSF, but also facilitates recovery of patients suffering from the deficiency of leukocytes and erythrocytes caused by anti-cancer drug treatment, and thus, can be used in combination with an anti-cancer drug as a chemotherapeutic adjuvant.

### SUMMARY OF THE INVENTION

An object of the present invention, therefore, is to provide an oral composition for enhancing the proliferation and differentiation of the osteoprogenitor cells comprising, as an active component, an extract of Dioscorea sp.

Another object of the present invention is to provide an oral composition for use as an anti-cancer drug adjuvant, which comprises, as an active component, an extract of Dioscorea sp.

A further object of the present invention is to provide an oral composition useful for the prevention and treatment of osteoporosis.

Still a further object of the present invention is to provide an oral composition useful as an adjuvant for the alleviation of the side effects caused by an anti-cancer drug.

Still in another object, the present invention provides a method for the treatment of osteoporosis comprising administering orally to a patient in need of the treatment an effective amount of a pharmaceutically active extract of Dioscorea sp.

Still in a further object, the present invention provides a method for alleviation of the side effects caused by chemotherapy, using a chemotherapeutic adjuvant, comprising administering orally to a patient treated with chemotherapy an effective amount of a pharmaceutically active extract of Dioscorea sp.

Other features and advantages of the present invention will become apparent in the following detailed description of the preferred embodiment with reference to the accompanying drawings.

### BRIEF DESCRIPTION OF THE DRAWINGS

FIG. 1 shows bar graphs illustrating the effects of (A) the methanol extract of Dioscorea sp and DHEA and (B) each further extracted fraction on the proliferation of osteoprogenitor cells of C3H mice;

FIG. 2 includes bar graphs showing the effects of (A) the methanol extract of Dioscorea sp and (B) each further extracted fraction on the differentiation of osteoprogenitor cells into matured osteoblastic cells of mice in vitro determined by alkaline phosphatase (ALP) activity;

FIG. 3 is a bar graph showing the in vitro effects of the methanol extract of Dioscorea sp on alkaline phosphatase (ALP) activity of the bone marrow cells derived from a patient suffering from glucocorticoid-induced osteoporosis;

FIG. 4 includes bar graphs showing the in vivo effects of the methanol extract of Dioscorea sp on (A) the differentiation of osteoprogenitor cells into matured osteoblastic cells of mice determined by alkaline phosphatase (ALP) activity, and on (B) the mineralization of the bone mass in the bone marrow cells of the healthy mice determined by the nodule formation;

FIG. 5 includes bar graphs showing the in vivo effects of the methanol extract of Dioscorea sp on (A) alkaline phosphatase (ALP) activity, and on (B) the mineralization of the bone mass in the bone marrow cells of ovariectomized mice;

FIG. 6 shows the RT-PCR result of cytokines in bone marrow cells isolated from mice which are orally administered with the methanol extract of Dioscorea sp. according to the present invention;

FIG. 7 consists of phase contrast micrographs showing the effect of methanol extract of Dioscorea sp. on morphological changes of the primary cultured mouse bone marrow cells in the presence of epithelial growth factor (EGF);

FIG. 8 shows the in vivo effect of the methanol extract of Dioscorea sp. on the leukocyte count in the peripheral blood of leukopenia mice induced by cyclophosphamide (CY);

FIG. 9 is a bar graph showing the in vivo effect of the methanol extract of Dioscorea sp. on red blood cell (RBC) count in the peripheral blood of cyclophosphamide (CP)-induced leukopenic mice suffering from severe anemia; and

FIG. 10 is a bar graph showing the in vivo effect of the methanol extract of Dioscorea sp. on hemoglobin content in the peripheral blood of cyclophosphamide (CP)-induced leukopenic mice suffering from severe anemia.

### DETAILED DESCRIPTION OF THE PREFERRED EMBODIMENT

The present invention is based on the discovery of the biological activities of the extract of Dioscorea sp., in particular the activity on cell regeneration. It was confirmed by experiments that the methanol- or ethanol-extract and the further extracted fractions of Dioscorea sp. prepared in accordance with the method of this invention include active substances that enhance the proliferation and differentiation of mouse bone marrow progenitor cells. Specifically, the methanol- or ethanol-extract and the further extracted fractions of Dioscorea sp. per se enhance cell proliferation of functional osteoprogenitor cells and even extensively induce the differentiation of osteoprogenitor cells into osteoblastic cells and enhance the mineralization of osteoblastic cells. Moreover, the methanol extract of Dioscorea sp. can alleviate the side effects caused by an anti-cancer drug. Specifically, the methanol extract restores the leukocytes and erythrocytes present in the peripheral blood of the mice treated with cyclophosphamide (CY.) Therefore, the methanol extract of Dioscorea sp. would be useful in the prevention and treatment of osteoporosis, a common disease in the aging process, and may be used in combination with an anti-cancer drug as an chemotherapeutic adjuvant.

Stem cell refers to those cells capable of self-renewal and differentiation. Stem cells are present at the maximal level during the embryonic period, and gradually decrease in number with aging. Thus, it was speculated that there is an important correlation/association between stem cells and aging. The stem cells in adults can generate a specific response toward a specific message transmitted through a microenvironment change generating new stem cells or differentiating into specific cells. When the stem cells receive a differentiation message, the stem cells rapidly reproduce in large amounts, and then finally proceed to differentiation. These stem cells are used for maintaining the balance of cells in adults, and replenish the number of cells that dies due to natural causes or injuries.

The stem cells in bone marrow are divided into two types, the hematopoietic stem cells which produce two more specialized types of stem cells, a lymphoid progenitor cells (which give rise to T and B lymphocytes) and a myeloid progenitor cells (which give rise to leukocytes, erythrocytes, and megakaryocytes), and stromal cells which are the source of the cells making up the supporting structure in the bone marrow. The stromal cells have the characteristic of adhering to the bottom of plastic culture plates during culturing, and can differentiate into osteoblasts, chondrocytes, adipocytes, and even myoblasts. Stromal cells are required for the growth and differentiation of hematopoietic stem cells.

The production and number of stem cells will be greatly reduced as aging occurs, leading to various problems of aging, in which osteoporosis is the most common. The causes of osteoporosis include the loss of balance between bone formation and resorption. The osteoblastic cells derived from the osteoprogenitor cells are responsible for bone formation consisted of the formation of the bone matrix and skeletal mineralization. Osteoprogenitor cells come from the stromal cells in the bone marrow. Dexamethasone and ascorbic acid can promote the proliferation and growth of osteoprogenitor cells, and enable the cells to differentiate into mature osteoblasts. During the differentiating process, different markers of osteoblasts are expressed: There is the deposition of collagenous matrix first, and after 10 to 14 days, alkaline phosphatase (ALP) is expressed. Alkaline phosphatase is widely used as a biochemical marker for identification of osteoblast activity, but its actual function is yet unknown, though currently it is believed that it participates in the skeletal mineralization process. After continuous culture to 21 days, the cells will secrete osteocalcein, and finally mineralize to form bone nodules.

In the present invention, the inventor unexpectedly found that the oral composition comprising a methanol- or ethanol-extract of Dioscorea sp. or the further extracted fractions can be used to enhance the proliferation and differentiation of osteoprogenitor cells in the absence of any bone cell modulating agent, and thus, the composition comprising the active extract can be used in the treatment of osteoporosis.

In the present invention, an oral composition for enhancing the proliferation and differentiation of cells comprises as an active component an extract of Dioscorea sp. The extract is prepared from the root tuber part of Dioscorea sp. and is obtained using alcohol-based solvent as the extracting solution. The preparation process involves (a) extracting a tuber of Dioscorea sp. with an alcohol-based solvent in the presence of an acid, preferably in the presence of 1% acetic acid, in which the alcohol-based solvent is methanol-, ethanol-based solvent, or mixture thereof.

In addition, the obtained extract may be further extracted on the basis of polarity, so as to obtain pharmacologically active fractions. In one preferred embodiment of this invention, the methanol extract is further subjected to partition chromatography comprising the steps of:

(b) using a solvent mixture of ethyl acetate and water to extract the methanol extract obtained from step (a) so as to separate the ethyl acetate from the water extract existing in the water phase;

(c) adding n-butanol solvent into the water phase to perform further extraction so as to separate a butanol extract from the remainder of water extract remaining in the water phase; and

(d) adding 75% alcohol solvent into the water phase obtained from step (c) to extract and further remove polysaccharide so as to obtain a purified water extract.

To confirm the biological activity of components of Dioscorea sp., analysis on the biological activity of the methanol extract of Dioscorea tuber and the further extracted fractions were conducted on cells obtained from normal mice and a human patient suffering from glucocorticoid-induced osteoporosis.

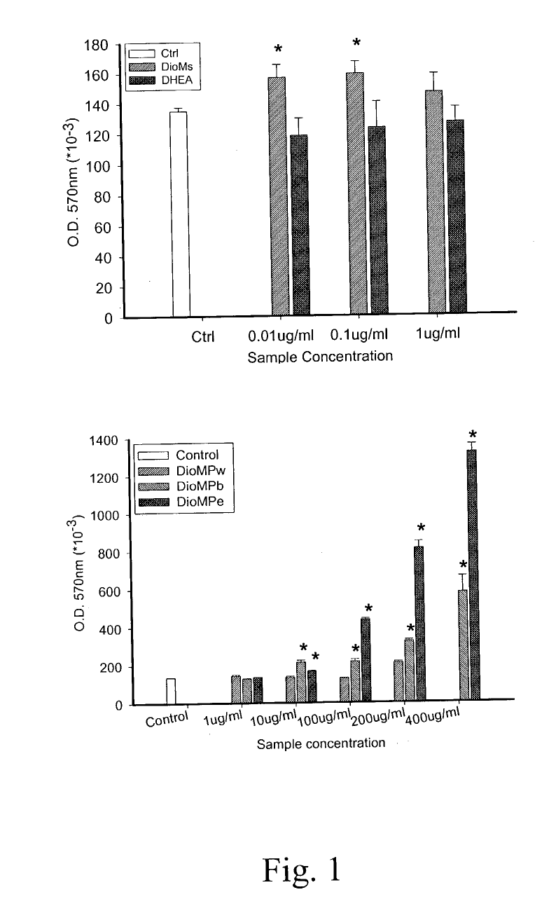

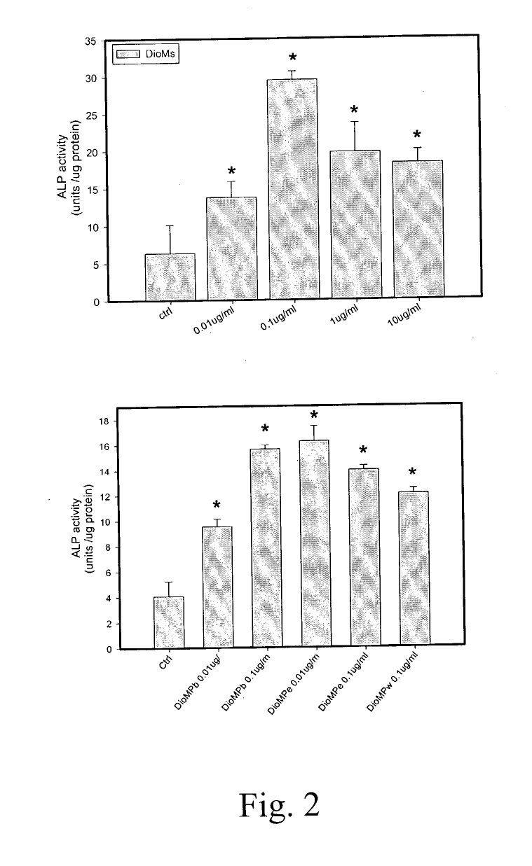

From the experimental results shown in FIG. 1, the inventor unexpectedly found that, the methanol extract of Dioscorea sp. and the further extracted fractions enhance the proliferation of the osteoprogenitor cells without the aid of any bone cell modulating agent. Under the same concentrations, DHEA exhibits no effect of enhancing the proliferation of osteoprogenitor cells. In FIG. 2, the result showed that the extracts of Dioscorea sp. significantly increase the amount of the expressed alkaline phosphatase in normal cells, that is, the extracts prepared according to the present invention can stimulate the differentiation of osteoprogenitor cells into mature osteoblastic cells.

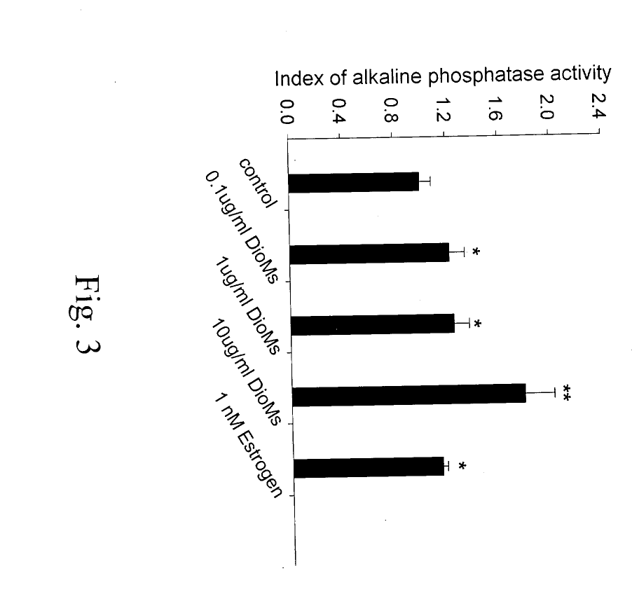

The inventor further confirmed the effect on abnormal bone marrow cells derived from a human patient suffering from glucocorticoid-induced osteoporosis. Glucocorticoids are essential therapies for a variety of inflammatory and autoimmune diseases. However, prolonged glucocorticoid use is one of the most common iatrogenic causes of osteoporosis. Glucocorticoids may increase bone loss through a variety of effects on osteoblasts, i.e., inhibition of the replication of the osteoblastic lineage, reduction of the genesis of new osteoblastic cells, and inducement of the death of osteoblastic cells. From the results shown in FIG. 3, the inventor found that the active extract of the present invention increases the amount of the expressed alkaline phosphatase in such cells, and thus, the function of osteoblastic cells may be recovered by treating a patient suffering from glucocorticoid-induced osteoporosis with the active extract.

Moreover, in order to further confirm the effectiveness of the extract of Dioscorea sp. in treating osteoporosis in vivo, the inventor conducts experiments on normal mice and the mice that are ovariectomized to induce osteoporosis, which were orally administered with the extract of the present invention.

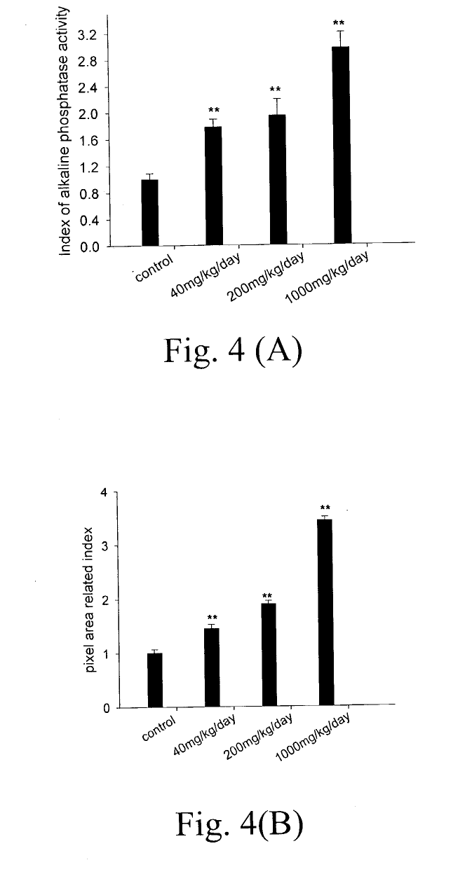

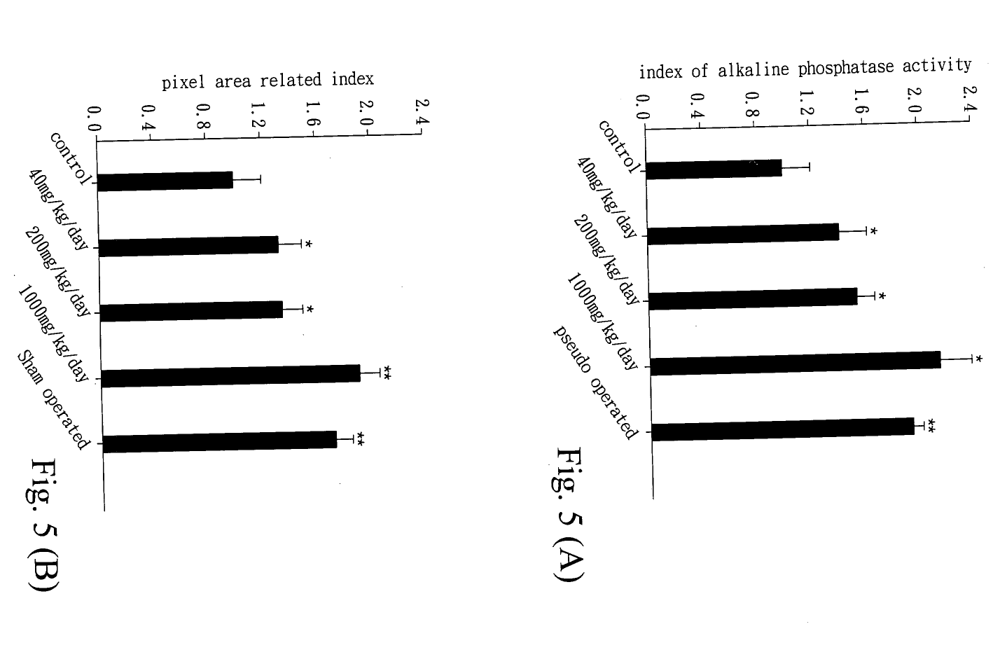

In FIGS. 4-5, the results demonstrate that the methanol extract of Dioscorea sp., in vivo, increases the amount of the expressed alkaline phosphatase and the mineralization of the osteoblastic cells derived from normal mice and ovariectomized mice. Thereofore, the extract of Dioscorea sp. not only regulates the proliferation and differentiation of osteoprogenitor cells but controls bone formation and remodeling, and thus, the active extract can prevent and treat osteoporosis.

On the other hand, it was known that the proliferation and differentiation of the bone marrow cells would be governed by certain factors, such as BMP-2 (bone morphogenetic protein-2,) TGF-β, IL-4, EGF, GM-CSF, etc. When the factor in the culture environment is changed, the stem cells differentiate into different cells in accordance with the specificity of such factors. For example, BMP-2, TGF-β, IL-4, and EGF are positively relevant to the proliferation and differentiation of the bone narrow stromal cells toward osteoblast lineage. In this study, the inventor conducted experiments to confirm the effects of the methanol extract of Dioscorea sp. on gene expression of BMP-2, TGF-β, and IL-4, and the effects of the methanol extract and the further extracted fractions of Dioscorea sp. on the proliferation and differentiation of bone marrow stem cells in the presence of EGF and GM-CSF.

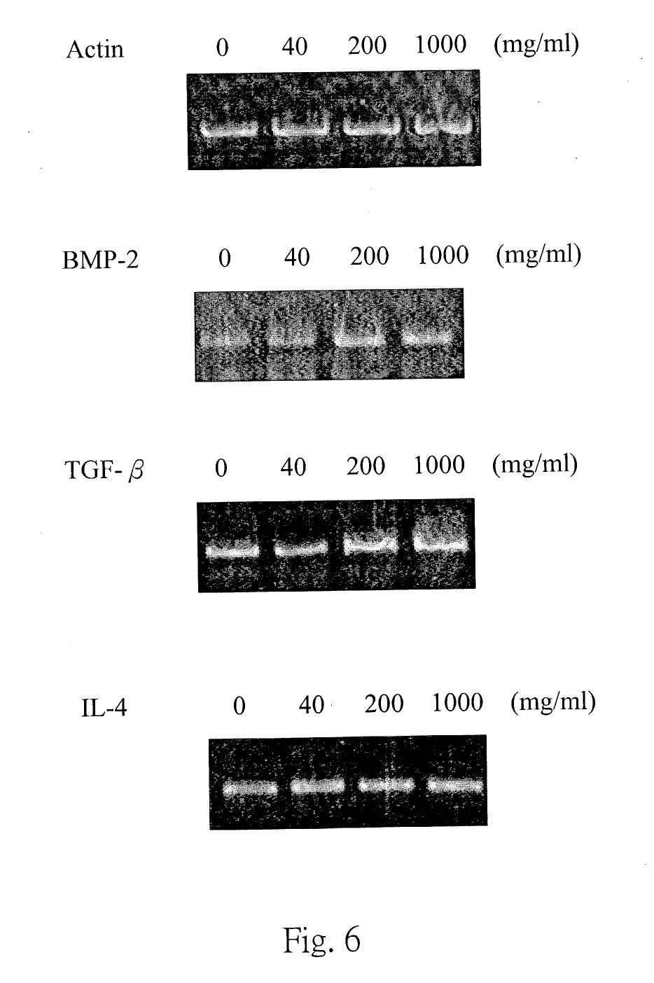

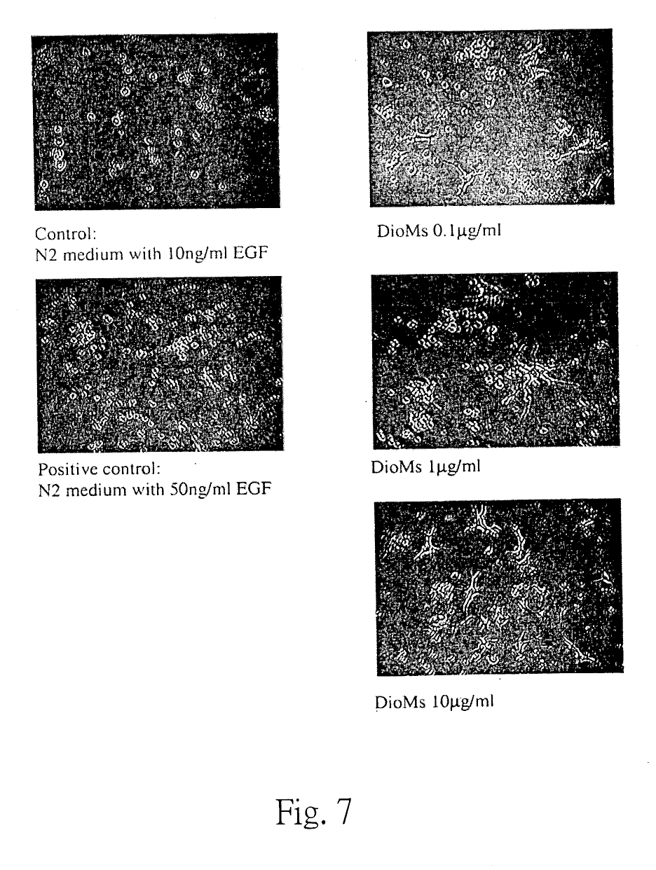

In FIG. 6, the data demonstrated that the the methanol extract of Dioscorea sp. increases the gene expression of BMP-2, TGF-β, and IL-4, especially that of BMP-2 and TGF-β. Moreover, from the experimental results shown in FIG. 7 and Table 2, the inventor found that the methanol extract of Dioscorea sp. stimulates the differentiation of the mouse bone marrow cells in the presence of EGF. The further extracted fraction of the methanol extract, DioMPw, enhances the proliferation of the mouse bone marrow cells.

As to GM-CSF, GM-CSF can act on a specific receptor complex present on hematopoietic progenitor cells to stimulate myelopoiesis, and thus, can promote the proliferation and differentiation of the hematopoietic progenitor cells in the bone marrow into monocytes, neutrophils, macrophages, etc. Therefore, it is believed that GM-CSF has potential in therapy for restoring macrophages of a patient treated with chemotherapy. In this study, the inventor found that, in the presence of GM-CSF, the proliferation of the bone marrow cells was enhanced under the stimulation of the methanol extract and the further extracted fractions of Dioscorea sp. (see Table 3). In addition, the result shows that the differentiation of stem cells was enhanced by the further extracted fractions of Dioscorea sp. Under the same condition, DHEA exhibits the effect of enhancing cell differentiation, but cannot enhance cell proliferation. Therefore, this study suggests that the methanol extract and the further extracted fractions of Dioscorea sp. prepared according to the present invention may assist GM-CSF in restoring the number of macrophages reduced by chemotherapy through the proliferation and proliferation of the bone marrow cells, and may be used as a chemotherapeutic adjuvant.

The inventor further confirmed the application of the extract of Dioscorea sp. on chemotherapy as a chemotherapeutic adjuvant in vivo. Cyclophosphamide (CY) is a drug used to treat a number of cancers; however, it destroys the bone marrow function, decreases the blood cells such as, leukocytes, macrophages, and erythrocyte, and causes many other side effects. In the present invention, cyclophosphamide is employed to cause leukopenia in mice to develop an animal model used to determine the function of the active extract of the present invention as a chemotherapeutic adjuvant. The obtained results showed that the active extract of the present invention prevented decrease of leukocyte count and maintained the red blood cell count and hemoglobin content to normal level, and thus, accelerated recovery from leukopenia in CY-treated mice. Therefore, the active extract of the present invention can be used as a chemotherapeutic adjuvant to alleviate the side effects induced by an anti-cancer drug.

The experiments performed in accordance with the present invention clearly demonstrate that the extract of Dioscorea sp. and the further extracted fractions thereof enhance the proliferation and differentiation of the osteoprogenitor cells in the absence of any bone cell modulating agent, and thus, the present invention provides an application of Dioscorea sp. in the treatment of osteoporosis. Moreover, the extracts prepared according to the present invention increase and restore the number of macrophages, leukocytes, and erythrocytes which has been reduced by chemotherapy, and can thus be used as a chemotherapeutic adjuvant.

The following examples are provided to illustrate the present invention. The examples are not intended to limit the scope of the present invention and they should be so interpreted.

### EXAMPLES OF THE INVENTION

### Preparatory Step 1

### Preparation of a Methanol Extract of Dioscorea sp.

4 kg Peeled tubers of Dioscorea sp. harvested from the Yang-Ming Mountain in Taiwan were immersed in 1% acetic acid solution overnight. The solid portion obtained was then frozen at −70° C. and lyophilized. The lyophilized portion was immersed in methanol solution in the presence of 1% acetic acid. After stirring and adjusting the concentration of methanol to 40% by volume, the mixture solution was allowed to stand overnight, and then separated by centrifugation. The obtained soluble fraction was lyophilized and is referred to as DioMs.

DioMs was further subjected to partition chromatography, comprising the steps of: using a solvent mixture of ethyl acetate and water (1:1) to extract DioMs so as to separate an ethyl acetate extract (referred to as DioMPe) from a water extract existing in the water phase; adding n-butanol solvent into the water phase to perform further extraction so as to separate a butanol extract (referred to as DioMPb) from the water extract remaining in the water phase; and adding 75% alcohol solvent into the water phase to extract and further remove polysaccharide so as to obtain a purified water extract (referred to as DioMPw).

### Preparatory Step 2

### Preparation of a Feed for Mice Containing a Methanol Extract of Dioscorea sp.

Purina Chow 5001, a commercially available mouse feed, was ground into powder. The lyophilized methanol extract of Dioscorea sp. were added in the ground feed in an amount to replace the same amount from the ground feed, to form a feed mixture. The feed mixture was uniformly mixed with distilled water, re-shaped by extrusion molding, baked for 2 min in microwave oven at proper power and, after cooling to room temperature, refrigerated at −70° C. After lyophilization, the feed mixture was formed into pellets very similar to the properties of the Purina Chow feed. The formed pellets are stored in −20° C freezer. The pellets are warmed to room temperature on the day of feeding, and are sterilized by UV lamp irradiation on a sterile work table. Feed mixtures having different concentrations of methanol extract were prepared.

### Preparatory Step 3

### Isolation and Culture of Bone Marrow Cells

Under sterile condition, SPF grade C3H/HeN mice were sacrificed and femoral bone was injected with a liquid culture of DMEM/F12 to flush out the bone marrow cells. The cells were filtered through sterile No. 53 nylon mesh. The single cell suspension so obtained is added with DMEM/F12 culture medium containing N2 to adjust to the proper concentration.

### Preparatory Step 4

### Preparation of Osteoprogenitor Cells from Mice

Under sterile condition, femoral bone of the SPF grade of C3H/HeN mouse was obtained and injected with DMEM/F12. The bone marrow cells were flushed out and filtered through No. 53 sterile nylon mesh. The single cell suspension obtained was added with DMEM/F12 culture medium containing 15% FCS to adjust the concentration of the cells.

The cells were cultured in DMEM/F12 medium containing 15% FCS, 50 μg/ml ascorbic acid, 10 mM sodium β-glycerophosphate and 10 nM dexamethasone in a T-flask for 6 days, the culture medium renewed every 3 days. The cell concentration was 10⁶ cells/cm². On the 6^{th} day, the suspended cells and culture medium were drawn out. The adhering cell layer was washed with 1×PBS that has been warmed to room temperature, and then treated with 0.01% EDTA at 37° C. for 5 to 10 minutes. The EDTA is removed and the reaction was stopped in the culture medium containing FCS. Treatment with TEG at 37° C. for 5 minutes followed. The reaction was similarly stopped in the culture medium containing FCS. The cells were all collected and centrifuged for 5 minutes at 1000 rpm.

### Example 1

### The Proliferative Response of Osteoprogenitor Cells of Mice Treated with the Methanol Extract and the Further Extracted Fractions of Dioscorea sp.

The cells obtained in preparatory step 4 were dispersed using a 22G gauge needle and suspended in DMEM/F12 medium containing 15% FCS, 50 μg/ml ascorbic acid, 10 mM sodium β-glycerophosphate and 10 nM dexamethazone to form a concentration of 4.5×10⁴ cells/ml. 225 μl cell suspension was added into each well of a 96-well microplate. After 3 hours, the cell suspension in each well was added with 25 μl methanol extract, each of the further extracted fractions and DHEA and incubated for 72 hours. Then, MTT assay was performed. 1 mg/ml MTT solution was added in each well and reacted for 4 hours. MTT lysis buffer (20% SDS-50% DMF) was added in each well in the amount of 150 μl/well and reacted for 16 hours, and the absorbance was measured at O.D. 570 nm for the resultant cell suspension in each well.

As shown in FIG. 1, under the stimulation of the methanol extract of Dioscorea sp., the proliferation of the osteoprogenitor cells was enhanced, in which the 0.01 and 0.1 μg/ml concentrations manifest significant enhancing effect. Under the same concentrations, DHEA exhibit no effect of enhancing the proliferation of osteoprogenitor cells. The extracted fractions, DioMPe and DioMPb, enhance cell proliferation at 10-400 μg/ml, wherein DioMPe exhibits an excellent effect.

### Example 2

### The Effect of the Methanol Extract and Different Extracted Fractions of Dioscorea sp on the Differentiation of Matured Osteoblastic Cells of Mice Determined by Alkaline Phosphatase Activity (ALP) In Vitro

Osteoprogenitor cells collected in preparatory step 4 were incubated in T-flask for 6 days. The cells were dispersed with 22G gauge needle, and the cell concentration was adjusted to 5×10³ cell/cm². The cells were then incubated in 6-well plates, in which 4.5 ml of cell culture medium was added in each well, and 0.5 ml methanol extract and each of the extracted fractions were added the next day. After incubation for 14 days, alkaline phosphatase activity assay was conducted thereon, as described below.

Drawing out the culture medium, the cell layer was washed several times with PBS. 0.5% Triton X-100 in PBS was added into each well. The resultant suspension is subjected to a freezing and thawing process at a temperature of −70° C. and 37° C. respectively. The treatment is done twice so as to obtain a test sample. 50 μl of the test sample was transferred from each well to an ELISA plate. 50 μl AMP-substrate buffer (2-amino-2-methyl-1-propanol (AMP, 0.5M) in distilled water, pH 10; 2 mM magnesium chloride and 9 mM p-nitrophenyl phosphate) was then added into the ELISA plate so as to be reacted with the test sample at room temperature for 10-20 min. Immediately after the absorbance was measured at 410 nm wavelength using ELISA reader, the protein concentration of each well was measured quantitatively. The measured alkaline phosphatase activity is expressed in unit/μg.

As shown in FIG. 2, after 14-day incubation of the bone marrow precursor cells, expression of alkaline phosphatase, which is the expressed marker specific to matured osteoblast, was noted. The methanol extract of Dioscorea sp. and each of further extracted fractions of the methanol extract significantly increase the amount of the expressed alkaline phosphatase, in which the methanol extract at 0.1 μg/ml, DioMPb at 0.1 μg/ml, DioMPe at 0.01-0.1 μg/ml, and the DioMPw at 0.1 μg/ml showed the strongest enhancement effect.

### Example 3

### The Effect of the Methanol Extract Dioscorea sp on Alkaline Phosphatase (ALP) Activity of Bone Marrow Cells Derived from a Patient Suffering from Glucocorticoid-Induced Osteoporosis

The patient's bone marrow cells obtained from the Taipei Veterans General Hospital were cultured in DMEM/F12 medium containing 15% FCS, 50 μg/ml ascorbic acid, 10 mM sodium β-glycerophosphate and 10 nM dexamethasone for 7 days. Alkaline phosphatase activity assay was conducted thereon.

As shown in FIG. 3, expression of alkaline phosphatase was noted. Compared with the control group and positive group (1 nM estrogen, which is the known treatment for osteoporosis,) the methanol extract of Dioscorea sp. increases the amount of the expressed alkaline phosphatase, in which the methanol extract at 10 μg/ml showed the strongest enhancement effect.

### Example 4

### The Effect of the Methanol Extract of Dioscorea sp on Alkaline Phosphatase (ALP) Activity and on Mineralization of Bone Marrow Cells In Vivo

Different concentrations (0, 40, 200, and 1000 mg/ml) of methanol extract were prepared for oral administration. After oral administration of different dosages by volume of the methanol extract for 5 days, mice were sacrificed to obtain bone marrow cells thereof.

(1) Alkaline Phosphatase Activity Assay

The bone marrow cells obtained were cultured in 96-well microplates at 2×10⁵ cells/well, in which 250 μl of α-MEM medium containing 15% FCS, 50 μg/ml ascorbic acid, 10 mM sodium β-glycerophosphate and 10 nM dexamethasone was added, and incubated in a 5% CO₂ incubator at 37° C. for 2 days. 125 μl/well of culture medium was drawn out and replaced by 125 μl/well of fresh medium containing 15% FCS, 50 μg/ml ascorbic acid, 10 mM sodium β-glycerophosphate, and 10 nM dexamethasone. After 4 days incubation, alkaline phosphatase activity assay was conducted thereon.

As shown in FIG. 4(A), expression of alkaline phosphatase was noted. In FIG. 4(A), the methanol extract of Dioscorea sp. increases the amount of the expressed alkaline phosphatase, in which the methanol extract at 1000 mg/kg might reach up to 3 times enhancement as compared with the control group.

(2) Nodule Formation Assay

This assay is used to analyze the mineralization of the bone mass. The bone marrow cells obtained from the mice orally administered with different concentrations (0, 40, 200, and 1000 mg/ml) of methanol extract were seeded in 24-well plates at 1×10⁶ cells/well, cultured in α-MEM medium containing 15% FCS, 50 μg/ml ascorbic acid, 10 mM sodium β-glycerophosphate and 10 nM dexamethasone, and incubated in a 5% CO₂ incubator at 37° C. for 24 hrs. 500 μl/well of culture medium was drawn out and replaced by 500 μl/well of fresh medium containing 15% FCS, 50 μg/ml ascorbic acid, 10 mM sodium β-glycerophosphate and 10 nM dexamethasone. The cells were further incubated for 15 days to analyze the mineralization of the bone mass, and the culture medium renewed every 4 days. Nodule formation assay was conducted thereon, as described below.

Drawing out the culture medium, the cells were fixed by reacting them with 500 μl/well formalin for 30 minutes in a 5% CO₂ incubator at 37° C. Removing formalin and rinsing the cells with sterilized water three times, 200 μl/well of 2% Alizarine Red S solution which reacts with calcium was added into the wells and the cells were further incubated in a 5% CO₂ incubator at 37° C. for 10 minutes. Then, Alizaine solution was removed and the cells were rinsed three times with absolute alcohol. The mineralized area of the bone mass was measured by Meta Image.

As shown in FIG. 4(B), compared with the control group, the methanol extract of Dioscorea sp. promotes mineralization of the bone mass, in which the methanol extract at 1000 mg/kg concentration showed the strongest enhancement effect of up to 3.5 times.

### Example 5

### The Effect of the Methanol Extract of Dioscorea sp on Alkaline Phosphatase (ALP) Activity and on Mineralization of Bone Marrow Cells in Ovariectomized Mice Model

Under sterile condition, a group of SPF grade C57BL/6j mice were subjected to a surgical operation to remove the ovary so as to induce the occurrence of osteoporosis and another group were merely operated on without removal of the ovary for use as a control group (referred to as pseudo operated mice).

Different concentrations (0, 40, 200, and 1000 mg/ml) of methanol extract were prepared for oral administration. After being orally administered with different dosages by volume of the methanol extract for 42 days, the mice were sacrificed to obtain bone marrow cells thereof.

(1) Alkaline Phosphatase Activity Assay

The bone marrow cells obtained from the mice orally administered with different concentrations (0, 40, 200, and 1000 mg/ml) of methanol extract and the pseudo operated mice were cultured in 96-well microplates at 2×10⁵ cells/well, in which 250 μl of α-MEM medium containing 15% FCS, 50 μg/ml ascorbic acid, 10 mM sodium β-glycerophosphate and 10 nM dexamethasone was added, and incubated in a 5% CO₂ incubator at 37° C. for 2 days. 125 μl/well of culture medium was drawn out and replaced by 125 μl/well of fresh medium containing 15% FCS, 50 ug/ml ascorbic acid, 10 mM sodium β-glycerophosphate and 10 nM dexamethasone was added. After 4 days incubation, alkaline phosphatase activity assay was conducted thereon.

As shown in FIG. 5(A), expression of alkaline phosphatase was noted. In FIG. 5(A), the methanol extract of Dioscorea sp. is shown to increase the amount of the expressed alkaline phosphatase, in which the methanol extract at 1000 mg/kg showed the strongest enhancement effect.

(2) Nodule Formation Assay

The bone marrow cells obtained from the mice orally administered with different concentrations (0, 40, 200, and 1000 mg/ml) of methanol extract and the pseudo operated mice were seeded in 24-well plates at 1×10⁶ cells/well, cultured in α-MEM medium containing 15% FCS, 50 μg/ml ascorbic acid, 10 mM sodium β-glycerophosphate and 10 nM dexamethasone, and incubated in a 5% CO₂ incubator at 37° C. for 24 hrs. 500 μl/well of culture medium was drawn out and replaced by 500 μl/well of fresh medium containing 15% FCS, 50 μg/ml ascorbic acid, 10 mM sodium β-glycerophosphate and 10 nM dexamethasone. The cells were further incubated for 15 days to analyze the mineralization of the bone mass, and the culture medium was renewed every 4 days. Nodule formation assay was conducted thereon.

As shown in FIG. 5(B), compared with the control group, the methanol extract of Dioscorea sp. promotes mineralization of the bone mass, in which the methanol extract at 1000 mg/kg showed the strongest enhancement effect.

### Example 6

### The Effect of the Methanol Extract Dioscorea sp on Gene Expression of Cytokines in the Bone Marrow Cells

Total RNA was extracted from the bone marrow cells obtained from Example 4 using Ultraspec™ RNA isolation kit (Biotex laboratories INC, U.S.A.). 5 μg total RNA and 2.5 μg oligo dT were heated at 70° C. for 10 minutes, cooled to room temperature for 10 minutes, then added with 4 μl 10 mM dNTP, 0.5 μl rRNasin, 1 μl (10 units) AMV (Avian Myeloblastosis virus) reverse transcriptase and the buffer thereof, and the final reaction volume was 26.5 μl. cDNA was obtained by reacting the previous reaction solution at 42° C. for 60 minutes and then at 90° C. for 5 minutes. 2.5 μl resultant cDNA was added with 0.5 μl 10 mM dNTP, 0.5 μl polymerase (2 units) and the buffer thereof, 1 μl of 10 μM targeted primers, and the final volume of the reaction mixture was 25 μl. PCR was performed for proper cycles, each cycle consisting of 45 seconds of denaturation at 94° C., 45 seconds of annealing at proper annealing temperature and 1 min of extension at 72° C. The reaction products were visualized by electrophoresis in 2% agarose gel. Sequences of the PCR primers were shown in Table 1. The experimental results are shown in FIG. 6.

As shown in FIG. 6, the gene expressions of BMP-2, TGF-β, and IL-4 are increased, especially BMP-2 and TGF-β.

TABLE 1
Sequences of the primers used in RT-PCR

| | | |
|---|---|---|
| Cytokine | Sequence (5′ to 3′) | Size (bp) |

| | | |
|---|---|---|
| IL-4 | | |
| sence | ATG GGT CTC AAC CCC CAG CTA GT | |
| antisence | GCT CTT TAG GCT TTC CAG GAA GTC | 399 |
| TGF-β | | |
| sence | TGG ACC GCA ACA ACG CCA TCT ATG CCA TCT ATG AGA AAA CC | |
| antisence | TGG AGC TGA AGC AAT AGT TGG TAT CCA GGG CT | 525 |
| BMP-2 | | |
| sence | CAT CCA GCC GAC CCT TG | |
| antisence | CTC TCC CAC TGA CTT GTG | 505 |
| β-actin | | |
| sence | GAC TAC CTC ATG AAG ATC CT | |
| antisence | CCA CAT CTG CTG GAA GGT GG | 510 |

### Example 7

### Effects of Methanol Extract of Dioscorea sp. on Morphological Changes of Bone Marrow Cells of C3H Mice in the Presence of Epithelial Growth Factor (EGF)

1×10⁴ cells/well bone marrow cells of mice obtained in preparatory step 3 were seeded in a 96-well microplate containing DMEM/F12 culture medium with N2 and 10 ng/ml EGF and incubated in a 5% CO₂ incubator at 37° C. for 24 hrs. Methanol extract and DHEA were added into different wells respectively. Wells containing pure culture medium into which no methanol extract and DHEA were added, and which were subjected to the same incubation procedure, were used as control. In addition, for use as positive control, the same procedure as that for control was repeated except that 50 ng/ml EGF was used. The proliferation responses were measured by virtue of MTT assay. In accordance with the MTT assay, each of the wells is added with 1 mg/ml MTT solution and, after 4 hours of reaction, MTT lysis buffer (20% SDS-50%DMF) was added thereinto in an amount of 150 μg/well. The resultant mixture was allowed to be reacted for 16 hours. The absorbance was measured at O.D. 570 nm.

As shown in FIG. 7, in the event that the progenitor cells in bone marrow induced with EGF proliferate, the methanol extract of Dioscorea sp. was found to further stimulate the bone marrow progenitor cells to differentiate significantly. It can be noted that a more desirable optimum result was obtained when the concentration of the methanol extract used was at 10 μg/ml. It is further shown in Table 2 that when 10 μg/ml of DioMPw was used for stimulation, the cells exhibited significantly enhanced proliferative effect. In the event that 100 μg/ml of DioMPw was used, the proliferation rate of the cell might reach up to 1.9 times that of the control as manifested in Table 2.

TABLE 2

| | | | Proliferation | Morphologic |
|---|---|---|---|---|
| Group | Concentration | | Index^{a} | Change^{b} |
| Control | | | 1.00 | + |
| DHEA | 0.0001 | μg/ml | 0.99 | + |
| | 0.001 | μg/ml | 0.64 | + |
| | 0.01 | μg/ml | 0.65 | + |
| | 0.1 | μg/ml | 0.67 | ++ |
| | 1 | μg/ml | 0.70 | ++ |
| DioMPw | 10 | ng/ml | 1.09 | − |
| | 100 | ng/ml | 1.13 | − |
| | 1 | μg/ml | 1.17 | − |
| | 10 | μg/ml | 1.34* | − |
| | 100 | μg/ml | 1.90* | − |

^{a}Data was analyzed by Student's t-test, and “*” means P < 0.05
^{b}Morphologic change was observed by microscope.

### Example 8

### The Proliferative Response of Bone Marrow Cells of Mice Treated with the Methanol Extract and the Further Extracted Fractions of Dioscorea sp. in the Presence of GM-CSF

1×10⁴ cells obtained in preparatory step 3 were seeded in each well of the 96-well microplate, cultured in DMEM/F12 medium containing N2, and 4 ng/ml mGM-CSF. Methanol extracts of Dioscorea sp. with different concentrations were respectively added into the resultant cultured cells in the different wells. The resultant methanol extract containing cultured cells in different wells were then incubated in a 5% CO₂ incubator at 37° C. for 14 days. The cells cultured in DMEM/F12 medium containing N2 and 20 ng/ml mGM-CSF is used as positive control. MTT assay was performed. In this respect, 1 mg/ml MTT solution was added in each well so as to react with the cultured cells therein for 4 hours. MTT lysis buffer (20% SDS-50% DMF) was added in an amount of 150 μl/well. The resultant mixture was allowed to reacte for 16 hours. The absorbance was measured at O.D. 570 nm.

As shown in Table 3, under the stimulation of the methanol extract of Dioscorea sp. in concentrations ranging from 0.001 μg/ml to 1000 μg/ml, the proliferation rate of the bone marrow cells was significantly increased. It can be noted that in terms of the proliferative enhancement, the most preferable range of concentration for the methanol extract is from 0.01 to 1000 μg/ml. The fractions resulting from further extraction of the methanol extract are all found to have the ability to enhance cell proliferation. Among them, DioMPb and DioMPe were particularly found to have excellent effect on the enhancement of proliferation. In addition, the 20-week adult mouse bone marrow cells were found to be able to differentiate upon stimulation by the methanol extract of Dioscorea sp. at the concentration of 1-10 μg/ml. Amongst the extracted fractions of the methanol extract, which were extracted by a solvent mixture of water and ethyl acetate, or followed by use of butanol and 75% alcohol in sequence to extract the water extract, both the ethyl acetate extracted fraction and the 75% alcohol extracted fraction resulting from further extracting the water extract with the 75% alcohol, are found to accelerate the differentiation of 20-week of adult mouse bone marrow cells. Under the same condition, however, DHEA, in contrast, exhibits only the effect of enhancing, cell differentiation, and fails to enhance cell proliferation. Therefore, the extracts of Dioscorea sp. which exhibit excellent effect on both the regeneration and differentiation of stem cells may assist GM-CSF in restoring the number of macrophages reduced by chemotherapy, and may be used as a chemotherapeutic adjuvant.

TABLE 3

| | | | Proliferation | Morphologic |
|---|---|---|---|---|
| Group | Concentration | | Index^{a} | Change^{b} |
| Control | | | 1.00 | + |
| DioMs | 0.0001 | μg/ml | 1.08 | + |
| | 0.001 | μg/ml | 1.19* | + |
| | 0.01 | μg/ml | 1.68* | + |
| | 0.1 | μg/ml | 1.95* | + |
| | 1.0 | μg/ml | 1.78* | ++ |
| | 10 | μg/ml | 1.82* | +++ |
| | 100 | μg/ml | 1.49* | + |
| | 1000 | μg/ml | 1.39* | + |
| DioMPe | 0.01 | μg/ml | 0.98 | + |
| | 0.1 | μg/ml | 1.11 | ++ |
| | 1 | μg/ml | 1.35* | ++ |
| | 10 | μg/ml | 2.64* | +++ |
| | 100 | μg/ml | 0.83 | −^{c} |
| | 300 | μg/ml | 0.97 | − |
| DioMPb | 0.01 | μg/ml | 1.07 | + |
| | 0.1 | μg/ml | 1.23 | + |
| | 1 | μg/ml | 1.34 | + |
| | 10 | μg/ml | 1.41 | + |
| | 100 | μg/ml | 1.90* | + |
| | 300 | μg/ml | 2.02* | + |
| DioMPw | 0.0001 | μg/ml | 1.06 | + |
| | 0.001 | μg/ml | 1.08 | + |
| | 0.01 | μg/ml | 1.12 | ++ |
| | 0.1 | μg/ml | 1.18 | ++ |
| | 1 | μg/ml | 1.31* | +++ |
| | 10 | μg/ml | 1.56* | + |
| DHEA | 0.0001 | μg/ml | 0.99 | + |
| | 0.001 | μg/ml | 1.15 | + |
| | 0.01 | μg/ml | 1.04 | + |
| | 0.1 | μg/ml | 1.13 | + |
| | 1 | μg/ml | 1.11 | +++ |
| | 10 | μg/ml | 1.19* | + |

^{a}Data was analyzed by Student's t-test, and “*” means P < 0.05
^{b}Morphologic change was observed by microscope.
^{c}Bone marrow cells were found dead under high concentration of DioMPe.

### Example 9

### The Effect of the Methanol Extract on the Number of Leukocyte and Red Blood Cells and Hemoglobin Content in Peripheral Blood of Leukopenia Mice Induced by Cyclophosphamide

Different concentrations (0, 20, 100, and 500 mg/ml) of methanol extract were prepared for oral administration. The mice were intraperitoneally injected with 200 and 100 mg/kg of cyclophosphamide (CY) on day 0 and day 5 to cause leukopenia, and blood was drawn periodically to cause anemie condition. The mice were orally administered with different dosages by volume of the methanol extract on day 1 until the mice were sacrificed. The peripheral blood collected from retro-orbital sinus was sampled on days 0,4, 8, and 12.

The blood (0.1 ml) obtained on days 0, 4, 8, and 12 was added with 25 μl EDTA solution (72 mg/ml) to prevent blood coagulation, and diluted 10× or 20× with Turk's solution (2% acetic acid with 0.01% crystal violet). The number of leukocytes counted by microscope.

The blood (0.1 ml) obtained on day 8 was added with 25 μl EDTA solution (72 mg/ml) to prevent blood coagulation, and diluted 2000× with saline. The number of erythrocytes was counted. Hemoglobin (Hbg) content was determined as described by Worthington R E et al., Experimental Hematology, 15:85-92, 1987.

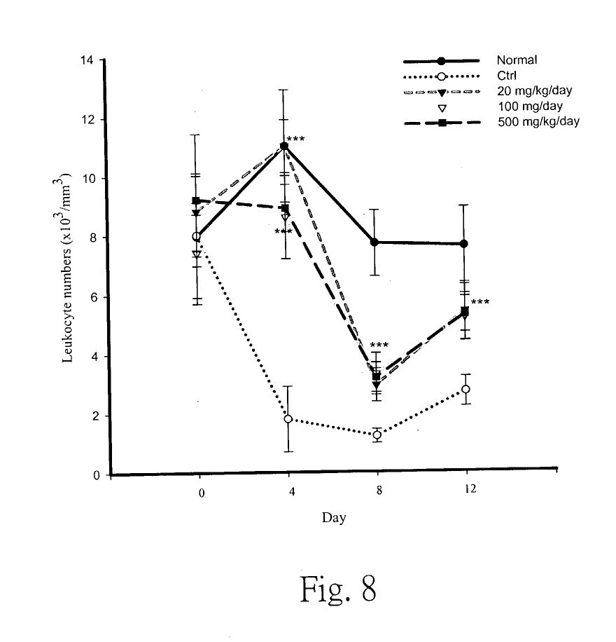

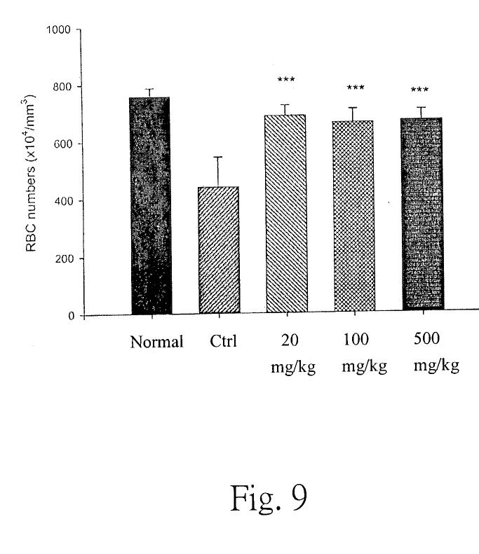

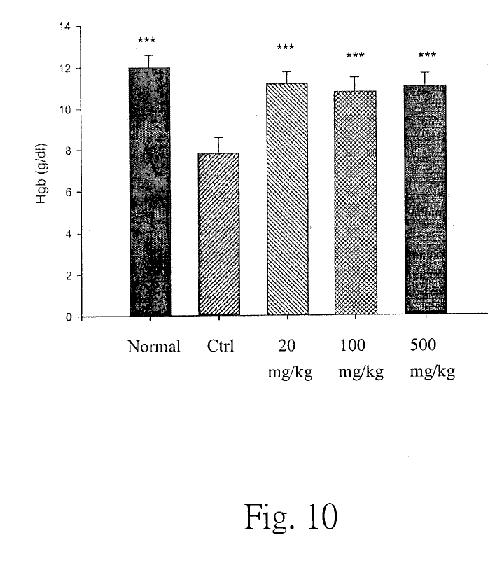

From the results shown in FIGS. 8-10, it is noted that the active extract of the present invention mitigates reduction of leukocyte number in peripheral blood and accelerates recovery of leukocytes, and maintains the red blood cells and Hbg content to normal level.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A method for treatment of osteoporosis comprising administering orally to a patient in need of such treatment an effective amount of a pharmaceutical composition containing as active component an extract of Dioscorea sp.

2. The method according to claim 1, wherein said extract is prepared by a process comprising the step of extracting a tuber of Dioscorea sp. with an alcohol-based solvent in the presence of an acid.

3. The method according to claim 2, wherein said alcohol-based solvent used in the preparing process is methanol, ethanol based solvent, or mixture thereof.

4. The method according to claim 2, wherein said acid used in the preparing process is 1% acetic acid.

5. The method according to claim 1, wherein said extract is prepared by a process comprising the steps of (a) extracting a tuber of Dioscorea sp. with an alcohol-based solvent in the presence of an acid, and (b) using a solvent mixture of ethyl acetate and water to extract said extract obtained from step (a) so as to separate said ethyl acetate extract from said water extract existing in the water phase.

6. The method according to claim 1, wherein said extract is prepared by a process comprising the steps of (a) extracting a tuber of Dioscorea sp. with an alcohol-based solvent in the presence of an acid, (b) using a solvent mixture of ethyl acetate and water to extract said extract obtained from step (a) so as to separate said ethyl acetate extract from said water extract existing in the water phase, and (c) adding n-butanol solvent into said water extract obtained from step (b) to perform further extraction so as to separate a butanol extract from the water extract remaining in the water phase.

7. The method according to claim 1, wherein said extract is prepared by a process comprising the steps of (a) extracting a tuber of Dioscorea sp. with an alcohol-based solvent in the presence of an acid, (b) using a solvent mixture of ethyl acetate and water to extract the extract obtained from step (a) so as to separate said ethyl acetate extract from said water extract existing in the water phase, (c) adding n-butanol solvent into said water phase obtained from step (b) to perform further extraction so as to separate a butanol extract from the water extract remaining in said water phase, and (d) adding 75% alcohol solvent into said water phase obtained from step (c) to extract and further remove polysaccharide so as to obtain a purified water extract.

8. A method for alleviation of side effects caused by chemotherapy, comprising administering orally to a patient treated with chemotherapy an effective amount of a pharmaceutical composition, as a chemotherapeutic adjuvant, containing an extract of Dioscorea Sp.

9. The method according to claim 8, wherein said extract is prepared by a process comprising the step of extracting a tuber of Dioscorea sp. with an alcohol-based solvent in the presence of an acid.

10. The method according to claim 9, wherein said alcohol-based solvent used in the preparing process is methanol, ethanol based solvent, or mixture thereof.

11. The method according to claim 9, wherein said acid used in the preparing process is 1% acetic acid.

12. The method according to claim 8, wherein said extract is prepared by a process comprising the steps of (a) extracting a tuber of Dioscorea sp. with an alcohol-based solvent in the presence of an acid, and (b) using a solvent mixture of ethyl acetate and water to extract said extract obtained from step (a) so as to separate said ethyl acetate extract from said water extract existing in the water phase.

13. The method according to claim 8, wherein said extract is prepared by a process comprising the steps of (a) extracting a tuber of Dioscorea sp. with an alcohol-based solvent in the presence of an acid, (b) using a solvent mixture of ethyl acetate and water to extract said extract obtained from step (a) so as to separate said ethyl acetate extract from said water extract existing in the water phase, and (c) adding n-butanol solvent into the water phase obtained from step (b) to perform further extraction so as to separate a butanol extract from said water extract remaining in the water phase.

14. The method according to claim 8, wherein said extract is prepared by a process comprising the steps of (a) extracting a tuber of Dioscorea sp. with an alcohol-based solvent in the presence of an acid, (b) using a solvent mixture of ethyl acetate and water to extract said extract obtained from step (a) so as to separate said ethyl acetate extract from said water extract existing in the water phase, (c) adding n-butanol solvent into the water phase obtained from step (b) to perform further extraction so as to separate a butanol extract from the water extract remaining in the water phase, and (d) adding 75% alcohol solvent into the water phase obtained from step (c) to extract and further remove polysaccharide so as to obtain a purified water extract.


SEQUENCE LISTING (8 sequences)

SEQ ID NO: 1 - DNA, 23 bp, Artificial Sequence
  Description of Artificial Sequence Primer
atgggtctcaacccccagctagt

SEQ ID NO: 2 - DNA, 24 bp, Artificial Sequence
  Description of Artificial Sequence Primer
gctctttaggctttccaggaagtc

SEQ ID NO: 3 - DNA, 41 bp, Artificial Sequence
  Description of Artificial Sequence Primer
tggaccgcaacaacgccatctatgccatctatgagaaaacc

SEQ ID NO: 4 - DNA, 32 bp, Artificial Sequence
  Description of Artificial Sequence Primer
tggagctgaagcaatagttggtatccagggct

SEQ ID NO: 5 - DNA, 17 bp, Artificial Sequence
  Description of Artificial Sequence Primer
catccagccgacccttg

SEQ ID NO: 6 - DNA, 18 bp, Artificial Sequence
  Description of Artificial Sequence Primer
ctctcccactgacttgtg

SEQ ID NO: 7 - DNA, 20 bp, Artificial Sequence
  Description of Artificial Sequence Primer
gactacctcatgaagatcct

SEQ ID NO: 8 - DNA, 20 bp, Artificial Sequence
  Description of Artificial Sequence Primer
ccacatctgctggaaggtgg